(19) United States
(12) Patent Application Publication
(10) Pub. No.: US 2017/0370899 A1
(43) Pub. Date: Dec. 28, 2017

(54) Methods for Evaluating Asphalt Mix Compositions Containing Reclaimed Asphalt

(71) Applicant: Arizona Chemical Company, LLC, Jacksonville, FL (US)

(72) Inventors: Laurent Porot, Almere (NL); David Broere, Almere (NL)

(73) Assignee: Arizona Chemical Company, LLC, Jacksonville, FL (US)

(21) Appl. No.: 15/630,713

(22) Filed: Jun. 22, 2017

Related U.S. Application Data
(60) Provisional application No. 62/353,539, filed on Jun. 22, 2016.

(51) Int. Cl. G01N 33/42; G01N 25/00; C08L 95/00; G01N 3/18

(52) U.S. Cl. CPC G01N 33/42; G01N 3/18; G01N 25/00; C08L 95/00; C08L 2555/10; C08L 2555/34; C08L 2555/52

(57) ABSTRACT

Compositions including reclaimed asphalt may be optimized for performance that is comparable with asphalt compositions that do not contain any reclaimed asphalt. In a method to determine an optimal level of reclaimed asphalt for use with at least a rejuvenator, TSRST tests are carried out on test asphalt compositions with varying levels of reclaimed asphalt (RA) materials to obtain failure temperature and failure stress data for all compositions. The test results are compared to identify composition(s) with optimal amount of RA. The optimal asphalt composition containing the maximum amount of RA for comparable performance may have a failure temperature within±5% of the failure temperature of the reference composition, and a failure stress that may be equal to or greater than the failure stress of the reference composition.

### CROSS REFERENCE TO RELATED APPLICATIONS

NONE.

### FIELD

The present disclosure relates generally to asphalt mix compositions containing reclaimed asphalt, and methods of making thereof and evaluating the asphalt mix compositions.

### BACKGROUND

Economic and environmental constraints lead to the valuation of products throughout their entire life cycle. For the pavement industry, the focus is on the overall management of the natural resources, such as aggregate and binder. Reclaimed asphalt, which may include reclaimed asphalt pavement (RAP) and reclaimed asphalt shingles (RAS), is one of the most recycled and reusable materials in the world.

To have an effective recycling method, the addition of recycled material must not have an adverse effect on the quality of the finished asphalt mix composition product. Rejuvenating agents and/or additives have been developed to treat the reclaimed asphalt to produce reclaimed asphalt mix compositions with performance characteristics comparable to asphalt mix compositions containing virgin materials not containing reclaimed materials. Rejuvenating agents enable the use of higher levels of recovered, also referred to as reclaimed, materials, such as RAP and/or RAS. However, if excessive amounts of reclaimed material are used with insufficient amounts of the rejuvenating agents and/or additives, or if an incorrect type of additives, the composition may suffer from poor performance such as early cracking failure due to changes in chemical and physical properties of the aged and stiffened binder in the asphalt mix composition.

A need exists, as a result, for a method to evaluate asphalt mix compositions to determine the optimal amounts and types of reclaimed materials, rejuvenating agents and/or additives for use in asphalt mix compositions to obtain the desired performance.

### SUMMARY

A method for evaluating an asphalt mix composition is provided herein. The method includes providing a reference asphalt mix composition comprising aggregates and virgin bitumen in a ratio of 90:10 to 95:5 of aggregates to virgin bitumen. The method further comprises providing a test asphalt mix composition comprising aggregates, a reclaimed asphalt and a rejuvenating agent. The method includes optionally performing at least one of compaction test, creep stiffness modulus test and low temperature cracking susceptibility test. A TSRST test is performed on the test asphalt mix composition and the reference asphalt mix compositions to obtain failure temperature and failure stress data for all asphalt mix compositions. The method further comprises comparing the failure temperature and failure stress data of the reference and test asphalt mix compositions to evaluate whether the test asphalt mix composition has an optimal amount of reclaimed asphalt, wherein the test asphalt mix composition with the optimal amount of reclaimed asphalt is defined as having a failure temperature within±5% of the failure temperature of the first reference asphalt mix composition, and a failure stress that is equal to or greater than the failure stress of the first reference asphalt mix composition.

### BRIEF DESCRIPTION OF THE DRAWINGS

FIG. 1 is a graph illustrating a gradation curve of a reclaimed asphalt mix composition.

FIG. 2 is a graph illustrating a gradation curve of another reclaimed asphalt example.

FIG. 3 is a graph illustrating the impact of the addition of 70% RA with bio-based additive on stiffness modulus.

FIG. 4 is a graph showing the fracture temperature results.

FIG. 5 is a graph showing basic properties of the recovered.

FIG. 6 is a graph illustrating the Dynamic Shear Rheometer (DSR) results of extracted binders.

FIG. 7 is a graph illustrating the creep stiffness and m-value temperature results from bending beam rheometers of binders.

### DESCRIPTION

The following terms may be used throughout the specification and may have the following meanings unless otherwise indicated.

“Asphalt” refers to a composite material that may include a (bituminous) binder and aggregate, which is generally used for paving applications, Asphalt may be of various types, including but not limited to stone mastic asphalt, stone mix asphalt, soft asphalt, hot rolled asphalt, dense-graded asphalt, gap-graded asphalt, porous asphalt, mastic asphalt, and other asphalt types.

“Reclaimed material” or “reclaimed asphalt” may include reclaimed asphalt pavement (RAP), reclaimed asphalt shingles (RAS), reclaimed asphalt from plant waste, reclaimed asphalt from roofing felt, asphalt from other applications, and virgin asphalt not meeting specifications.

Aggregate (or “construction aggregate”) may be any particulate mineral material suitable for use in asphalt. It generally includes but not limited to sand, gravel, crushed stone, slag, granite, limestone, and mixture thereof Any conventional type of aggregate suitable for use in asphalt may be used herein.

“Virgin asphalt” refers to a combination of virgin aggregate with virgin bitumen or virgin binder. Virgin asphalt has not been used previously for paving.

“Rejuvenating agent” or rejuvenator refers to an additive, a composition, or a mixture that may be combined with aged binder or reclaimed asphalt, or mixtures thereof, with virgin binder and/or virgin asphalt, to revitalize the aged binder or reclaimed asphalt such that some or all of the original properties of virgin binder and/or virgin asphalt are restored.

“Tall oil” refers to any of man-made and naturally occurring tall oil, tall oil pitch, tall oil blends, and similar tall oil products. Tall oil is a liquid resinous material that may be obtained in the digestion of wood pulp from paper manufacture. Commercial tall oils include but not limited to a complex of fatty acids, resin acids, sterols, higher alcohols, waxes and hydrocarbons. The acid components may also be present as esters thereof.

In one embodiment, the disclosure relates to a method for optimizing asphalt mix compositions, also referred to herein as asphalt compositions, by evaluating the asphalt mix composition characteristics, and comparing the characteristics of the asphalt mix compositions to those of a reference sample, or samples, to maximize the amount of reclaimed material(s) in the asphalt mix composition with minimal adverse effects on the characteristics thereof. Examples of asphalt mix composition characteristics include but not limited to cracking susceptibility and elastic modulus at different temperatures. In some embodiments, asphalt composition evaluation is based on cyclic tests (complex modulus) and static low temperature tests (thermal stress restrained specimen test, referred to herein as “TSRST”). Binder evaluation may include determination of binder properties such as penetration and softening point temperature, and rheological properties using Dynamic Shear Rheometer and Bending Beam Rheometer.

In an aspect, an asphalt mix composition to be evaluated, including for example an asphalt composition for paving applications, may include aggregate(s), rejuvenating agent(s), reclaimed material(s), bitumen, and other optional materials, with the amount of reclaimed material(s) of at least 15 wt. %, alternatively 20 wt. %, alternatively 25 wt. %, alternatively with at least 50 wt. %, alternatively with 25 wt. % to 100 wt. %, alternatively with 50 wt. % to 100 wt. %, alternatively with 50 wt. % to 70 wt. % based upon the total asphalt mix composition.

In an aspect, an asphalt composition may include less than 50 wt. % aggregate(s). Aggregate may include any mineral material known in the art. Properties of aggregates may include cubic and/or angular shape (to interlock with other particles), durability (resistance to weathering), frictional resistance, hardness (enough to resist crushing, degradation and disintegration), hydrophobicity (to resist raveling and stripping), load transmittance, low reactivity with alkalis (to avoid an expansive reaction that can lead to cracking, surface popouts and spalling), resistant to weathering (such as wetting, drying, freezing and thawing), rough surface texture (skid resistance), soundness (resistance to weathering), strength and toughness (enough to resist crushing, degradation and disintegration).

Aggregate may be fractionated to produce a mixture of gradations. A mixture of gradations may allow smaller particles to pack between larger particles. Aggregates may be fractionated one or more times to achieve the desired particle size or sizes, resulting in aggregate particles less than 19.0 mm in diameter, less than 12.7 mm in diameter, less than 9.51 mm in diameter, less than 4.76 mm in diameter, less than 0.595 mm in diameter, less than 0.297 mm in diameter or less than 0.074 mm in diameter, respectively.

In an aspect, an asphalt mix composition, also referred to herein as an asphalt composition, of the present disclosure may include a rejuvenating agent, or rejuvenating agents, also referred to herein as additives. An asphalt mix composition may include bitumen. Bitumen typically stiffens and becomes brittle as it ages due to chemical changes within the binder. Changes to the properties may include loss of maltenes which act to soften and lower the viscosity of the binder—and an increase in asphaltenes and polar aromatics through oxidation, requiring the use of rejuvenation agent(s)/additive(s). Bitumen and bituminous binders are described in detail in EN 12591 standard including soft bitumen grades. Soft binders may include soft bitumen grades or petroleum based fluxes. To restore the properties of aged bitumen, additives are needed to rejuvenate the aged bitumen in the reclaimed materials.

In one embodiment, an asphalt composition may include a rejuvenating agent in an amount ranging from 0.010 to 20 wt. %, alternatively 0.1 to 15 wt. %, alternatively 1 to 10 wt. % based upon the total amount binder and agent. Rejuvenating agents may include naturally occurring compounds and synthesized chemical compounds including but not limited to surfactants, detergents, wetting agents and emulsifiers. Surfactant additives may be any of: i) anionic surface agents such as fatty acids, saturated and/or unsaturated fatty acids, fatty acid pitch such as stearic acid pitch, and fatty acid derivatives including but not limited to fatty acid esters and fatty acid sulfonates, organo-phosphates including but not limited to alkyl phosphates ii) cationic surface agents such as alkyl amines, alkyl quaternary ammonium salts, heterocyclic quaternary ammonium salts, amido amines, and non-nitrogenous sulfur or phosphorous derivatives; iii) ampholytic surface agents such as amino acids, amino acid derivatives, betain derivatives including but not limited to alkylbetains and alkylaminobetains, imidazolines, imidazoline derivatives; and iv) non-ionic surface agents having fatty acid ester bonds surfactants, including, but not limited to, SPANS and TWEENS, with ether bonds, alkylphenolpolyoxeythylenes and plyoxyethylenated alcohols, surfactants with amide bonds including but not limited to alkanolamides, mono and diethanolamides and their derivatives, alkylenated oxide copolymers and polyoxyethyleneated mercaptans. In one embodiment, the rejuvenating agent is an additive displaying an amphipathic chemical structure that serves to both disperse the highly polar fractions and to boost the solvating power of the maltene fraction even at a very low dosage.

Examples of rejuvenating agents include, but are not limited to compositions disclosed in U.S. Pat. No. 9,139,733; U.S. Pat. No. 8,034,172; US Patent Application No. 2014/0338565; US Patent Application No. 2015/0240081; US Patent Application No. 2016/0115352; and International Patent Publication No. WO 2016/054035, incorporated herein by reference in their entirety.

In some embodiments, a rejuvenating agent may be selected from the group consisting of trimethylolpropane tallates, ethylene glycol monomerates, neopentyl glycol monomerates, 2-ethylhexyl monomerates, and glycerol monomerates. In another embodiment, the rejuvenating agent may include an ester or ester blend derived from an acid selected from the group consisting of aromatic acids, fatty acids, fatty acid monomers, fatty acid dimers, fatty acid trimers, rosin acids, rosin acid dimers, and mixtures thereof, with a cyclic content of at least 5 wt. %. In yet another embodiment, the rejuvenating agent is an ester derived from tall oil fatty acids, monomer acids, and dimer acids.

In some embodiments, the rejuvenation agent is a bio-based material, including but not limited to tall oil materials, lecithin based materials, and mixtures thereof An example is a tall oil material is disclosed in U.S. Pat. No. 8,034,172, included herein by reference. Other examples of bio-based rejuvenators include resins derived from cashew nut shell liquid, as disclosed in U.S. Pat. No. 2,523,623, incorporated herein by reference.

In some examples, the rejuvenating agent is used in conjunction with an additive, such as a surfactant or a surfactant mixture. In other examples, a surfactant or a surfactant mixture is used instead of or as the rejuvenating agent. Examples of surfactants include sorbitan-based surfactants as disclosed in U.S. Pat. No. 8,039,046 incorporated herein by reference. In other examples, the surfactant may include an alkoxylated fatty amine as disclosed in EPO Patent Publication No. EP 2966128, incorporated herein by reference. In yet other embodiment, the surfactants are selected from non-ionic and cationic surfactants which comprise 2 or more identical or different heteroatoms, said heteroatoms being chosen from among nitrogen, oxygen, phosphorus and sulfur, with more than two identical or different heteroatoms, and at least two heteroatoms which are different from one another, as disclosed in US Patent Publication No. 2014/0230693, incorporated herein by reference.

In an aspect, an asphalt mix composition may include a reclaimed material. The asphalt mix composition may include at least 15 wt. %, alternatively at least 20 wt. %, alternative at least 25 wt. %, alternatively at least 50 wt. %, alternatively 25 wt. % to 100 wt. %, alternatively 50 wt. % to 100 wt. %, alternatively 50 wt. % to 70 wt. % of reclaimed asphalt based upon the asphalt mix composition. The reclaimed material may be “reclaimed asphalt pavement” (RAP) by itself, recycled asphalt singles (RAS) by itself, or a mixture of both RAP and RAS. RA may be obtained from asphalt that has been removed from the road or other structure, and then processed by well-known methods, including milling, ripping, breaking, crushing, and/or pulverizing. RAP is asphalt that has been used previously as pavement. Prior to use, the reclaimed asphalt material may be inspected, sized and selected, for instance, depending on the final paving application. As used herein, the term RAP refers to reclaimed asphalt pavement (RAP) itself, recycled asphalt singles (RAS), or a mixture of both.

In an aspect, an asphalt mix composition may further include optional materials, in some embodiments, the asphalt mix composition may include optional materials in an amount ranging from 0.10 to 10 wt. % based upon the total asphalt mix composition. Examples of optional materials include but are not limited to elastomers, non-bituminous binders, adhesion promoters, softening agents, phosphoric acid, fiber, hardening agent, or other suitable additives. Useful elastomers include, for example, ethylene-vinyl acetate copolymers, poly butadienes, ethylene-propylene copolymers, ethylene-propylene-diene terpolymers, butadiene-styrene block copolymers, styrene-butadiene-styrene (SBS) block terpolymers, isoprene-styrene block copolymers and styrene-isoprene-styrene (SIS) block terpolymers, or the like. Cured elastomer additives may include ground tire rubber materials.

In yet another aspect, an asphalt mix composition may include bitumen. Bitumen is generally used as a binder in the asphalt mix composition in an amount ranging from 0.1-10 wt. % based upon the asphalt composition. Bitumen is a mixture of viscous organic liquids or semi-solids from crude oil that is black, sticky, soluble in carbon disulfide, and composed primarily of condensed aromatic hydrocarbons. Alternatively, bitumen refers to a mixture of maltenes and asphaltenes. The bitumen may be naturally occurring. It may be crude bitumen, or it may be refined bitumen obtained as the bottom residue from vacuum distillation of crude oil, thermal cracking, or hydrocracking.

In one embodiment, bitumen may contain or may be obtained from reclaimed asphalt pavement, such as for example bitumen of RA origin. In another embodiment, bitumen is virgin bitumen, also referred to as “fresh bitumen”, or bitumen that has not been used before for roads and/or construction applications. Such bitumen has not been recovered from road pavement.

In some embodiments, the bitumen may comprise aged binder which is a binder that may be present in or may be recovered from reclaimed asphalt, or hard, poor-quality, or out-of-spec virgin binders. Aged binder has high viscosity compared with that of virgin bitumen as a result of aging and exposure to outdoor weather, or due to poor quality.

Also disclosed herein are methods for forming asphalt mix compositions for evaluation and optimization of the same. The asphalt mix compositions for evaluation to optimize RA amount may be formed by methods known in the art. In one embodiment, a hot mix method is employed, wherein the various components are first heated to different temperatures for a final asphalt composition at a temperature between 150° C.-200° C., alternatively 160° C.-180° C. In one example, the RA is heated to a temperature in the range of 120° C.-150°, alternatively in the range of 130° C.-140° C. The aggregate may be heated to a temperature in the range of 250-320° C. The bitumen is heated up to a temperature in the range of 160° C.-180° C. The optional materials may be heated up separately for subsequent mixing into other components or into the asphalt mix composition. In one embodiment, some or all of the optional materials are mixed into any of the bitumen, the aggregate, or added to the RA prior to or after heating, or mixed into the final asphalt mix composition.

In some embodiments, a warm mix method is employed to generate the asphalt mix compositions for optimization, wherein the various components are first heated to different temperatures in the range of 100° C.-140° C., with other components heated to temperatures as in the hot mix method. In such embodiments, the aggregate may be heated to a lower temperature in the range of 100° C.

In other embodiments, the asphalt mix composition may be prepared as a cold mix, with the formation of an asphalt emulsion. In such embodiments may be prepared by combining asphalt particles, water and surfactants, and the rejuvenating agent(s), using a high shear mechanical device. The asphalt emulsions may be subsequently mixed with the aggregates and the RA.

In yet another embodiment, a method to determine an optimal asphalt composition may include determining an asphalt composition with the most amount of RA that is acceptable such that the characteristics/performance properties of these asphalt mix compositions are comparable with asphalt mix composition containing little or no RA. First, a reference asphalt mix composition may be formed without addition of RA. The reference asphalt mix composition may contain virgin bitumen, an aggregate or aggregates, and optional materials. In one embodiment, the reference asphalt mix composition, also referred to herein as a virgin bitumen composition, may include aggregates in an amount of from 92 wt. % to 96 wt. %, bitumen in an amount of from 3 wt. % to 7 wt. %, and optional materials in an amount of from 0 wt. % to 3 wt. %. The amounts of the components disclosed herein are based upon the total reference asphalt mix composition. It should be noted that the reference asphalt mix composition may be formed such that its performance characteristics meet the standard or the criteria of a particular location where the optimized asphalt mix composition is to be applied.

The method to determine an optimal asphalt mix composition may further include forming another reference asphalt composition that may include RA. The second reference asphalt mix composition may contain the same type of aggregate, bitumen, and optional materials (if any) as in the reference asphalt mix composition containing virgin bitumen, and including the same amounts of aggregate(s), bitumen and optional materials. These amounts based upon the total second reference asphalt mix composition may be adjusted to accommodate the addition of RA. The second reference asphalt mix composition may be prepared by adding RA in an amount of at least 15 wt. %, alternatively at least 20 wt. %, alternatively at least 25 wt. %, alternatively at least 50 wt. %, alternatively 25 wt. % to 100 wt. %, alternatively 50 wt. % to 100 wt. %, alternatively 50 wt. % to 70 wt. %. In one embodiment, at least three reference asphalt mix compositions may be formed with different amounts of RA. For example, the reference asphalt mix compositions may include RAP in an amount of 0 wt. %, 15 wt. %, 20 wt. %, 25 wt. % , 50 wt. %, 65 wt. %, and 80 wt. % based upon the reference asphalt mix composition.

In aspect, the method to determine an optimal asphalt mix composition may further include forming at least another asphalt mix composition, referred to herein as a test asphalt mix composition. The test asphalt mix composition may be formed with RA in an amount of at least 15 wt. %, alternatively at least 20 wt. %, alternatively at least 25 wt. %, alternatively at least 50 wt. %, alternatively 25 wt. % to 100 wt. %, alternatively 50 wt. % to 100 wt. %, alternatively 50 wt. % to 70 wt. % based upon the total test asphalt mix composition. The test asphalt mix composition may further include a rejuvenator. The test asphalt mix composition may include the same type of aggregate, bitumen and optional materials as in the reference asphalt mix composition containing virgin bitumen, and the same type of RA as in the reference asphalt mix composition with RA, with all components prorated to accommodate the addition of the rejuvenator in an amount 0.10 to 20 wt. % based upon the combined amounts of binder and rejuvenator. In one embodiment, the test asphalt mix compositions may be formed with RA in differing amounts and with different rejuvenators wherein the rejuvenators may be present in different amounts such as 0.1 wt. %, 0.5 wt. %, 1 wt. %, 4 wt. %, 6 wt. %, and so on up to 20 wt. % based upon the combined amounts of binder and rejuvenator.

In another embodiment, compaction tests may be carried out with all reference and test asphalt mix compositions at different temperatures. In an aspect, the tests may be carried at two different temperature points, the first temperature being the temperature of a hot mix as delivered at a pavement site. The first temperature may be about 90° C.-135° C. The second temperature may be the temperature of a warm mix as delivered at a pavement site. The second temperature may be about 90° C.-115° C.

In an aspect, the methods disclosed herein may further include evaluating the reference and test asphalt mix compositions for high temperature resistance such as rutting performance, intermediate temperature behavior such as fatigue performance. The intermediate temperature may include temperatures in the range of 20° C.-40° C. These tests may generally be performed after compaction tests. In another aspect, low-temperature cracking susceptibility of the asphalt mix compositions may be evaluated using Thermal Stress Restrained Specimen Test (TSRST) according to EN 12697-46 standard test or AASHTO TP 10-93 test method. Referred to herein as the “Standard test method for Thermal Stress Restrained Specimen Tensile Strength (TSRST)” was published in 1993. In the TSRST, changes in the binder's glass transition temperature are observed in order to determine the effects of low-temperature cracking in the asphalt compositions. Each specimen is glued to a plate at each end. The test device cools the specimen while restraining it from contracting. As the temperature drops, thermal stresses build up and are allowed to increase until the asphalt mix composition specimen is fractured.

In the evaluation step, test results of all asphalt composition samples may be compared to determine which test asphalt mix composition contains an optimal amount of the reclaimed asphalt (RA). The asphalt mix composition with the optimal amount of RA and the rejuvenator will typically have recovered asphalt mix composition properties at intermediate temperatures, and restored flexibility at low temperatures. The recovered properties may include relaxation of the binder (where the binder becomes less stiff and brittle) and improved cracking susceptibility of the asphalt composition. In one embodiment, the test asphalt composition with the optimal amount of RA may have a failure temperature of within±5% of the failure temperature of a reference (virgin bitumen) composition without RAP, and with a failure stress that is equal or greater than the failure stress of the reference composition.

### EXAMPLES

The following illustrative examples are intended to be non-limiting.

### Example 1

A number of test asphalt mix compositions, also referred to herein as mixtures or asphalt mixtures, were generated and evaluated for properties including stiffness modulus and low temperature cracking susceptibility. The comparative (base mixture) did not have reclaimed asphalt and the second comparative mixture contained 70 wt. % of RA. The third mixture contained 70 wt. % of reclaimed asphalt (RA) and a rejuvenation agent. In the examples, the chosen asphalt mixture was an AC 16, 50/70 BN, according to the EN 13108-01 European standard. The rejuvenator, or rejuvenating agent added to the third asphalt mix composition was SYLVAROAD™ RP1000 performance additive commercially available from Arizona Chemical Company, LLC, a subsidiary of Kraton Corporation, used in an amount of 4 wt. % per weight of binder content from the RA. The rejuvenating agent remains liquid at ambient temperature having properties as shown in Table 1.

TABLE 1
Rejuvenating Agent Properties.

| Properties | Typical Values |
|---|---|
| Flash point | >280° C. |
| Viscosity at 60° C. | 22 cSt |
| Density | 0.93 |
| Cloud point | <−25° C. |

The different asphalt mix compositions were tested to determine an optimized asphalt mix composition such that the optimized asphalt mix composition having ≧70 wt. % of RAP had similar or improved performance characteristics compared to the asphalt composition without the reclaimed asphalt. The asphalt composition samples were evaluated to determine the low temperature performance using Thermal Stress Restrained Specimen Test (TSRST) as described above. The complex modulus for asphalt mix compositions having high RA was produced.

In one example, the reclaimed asphalt (RA) used had the properties illustrated in Table 2, with the RA binder extracted and recovered and characterized for penetration value at 25° C. according to EN1426 and softening point temperature according to EN 1427.

TABLE 2
Properties of Reclaimed Asphalt.

| | Binder | Penetration | Softening |
|---|---|---|---|
| | content | 25° C. | point 25° C. |
| Reclaimed | 4.94% | 29 dmm | 55.1° C. |
| Asphalt | | | |

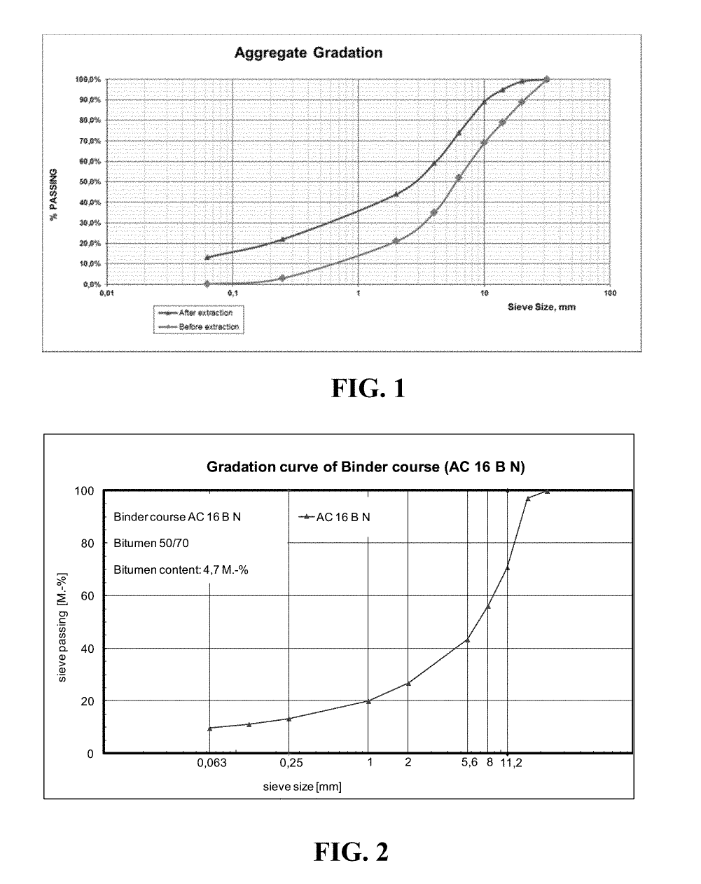

Grading curve for the RA was performed before and after the extraction as illustrated in FIG. 1, showing a difference in grading curves, where after the extraction the fine part was more relevant than before the extraction. The fine RA contains sand and fine particles. The rejuvenator may mobilize the binder from the RA.

The mechanical characteristics of the asphalt composition mixtures were first evaluated by determining stiffness modulus and low temperature cracking susceptibility. The asphalt composition mixtures were produced by using a standard opposite rotation pug mill according to the standard EN 12697-35. Mixing temperature was kept constant at 170° C. for all three asphalt composition mixtures. After mixing the compositions were stored in an oven at 135° C. In addition, one asphalt composition was kept at 95° C. before compaction to determine the effects on the final asphalt composition characteristics. Asphalt compositions and their components are shown in Table 3.

TABLE 3
Asphalt Compositions.

| | | | Reclaimed | | Compaction |
|---|---|---|---|---|---|
| | Mix | | asphalt | Rejuvenating | temperature |
| No | type | Bitumen | (mass-%) | Agent | (° C.) |
| 1 | AC 16 | 50/70 | 0 | No | 135 |
| 2 | | | 70 | No | 135 |
| 3a | | | 70 | Yes | 135 |
| 3b | | | 70 | Yes | 95 |

In the next step, compaction was carried out. Compaction was performed using a standard segment roller compactor according to EN 12697-33. Asphalt compositions and the compaction device were preheated to the compaction temperature. The compaction temperature for the asphalt compositions was 135° C. For the test asphalt composition (containing 70 wt. % RA and SYLVAROAD™ RP 1000) two compaction temperatures were used, the first temperature being 135° C. and the second temperature being 95° C. Prior to testing, homogeneity of the slabs was determined for, bulk density according to EN 12697-6 and air void content according to EN 12697-8.

Stiffness modulus was determined by using the cyclic indirect tensile test (CIDT) according EN 12697-24. A cyclic load was applied to the cylindrical asphalt specimen via two diametrically opposite load transfer rails. To fix the position of the asphalt specimen, a minimal stress of δᵤ=0.035 MPa is applied, while the maximum stress is chosen with regard to elastic horizontal strains between 50 and 100 μm/m. Tests were carried out for temperature frequency sweeps, with the experimental parameters shown in Table 4.

TABLE 4
CIDT Test Parameters.

| Asphalt layer | Binder Layer Material |
|---|---|
| Test temperature (° C.) | 20; 10; 0; −10 |
| Loading frequency (Hz) | 0.1; 1; 5; 10 |

For characterization of the low temperature cracking susceptibility, the prismatic asphalt specimen were subjected to Thermal Stress Restrained Specimen Tests (TSRST) in accordance to EN 12697-46. During the TSRST, the specimen were held at constant length, while temperature was decreased from 20° C. to −40° C. with a constant cooling rate of −10 K/h. The test ended either at the minimum test temperature of −40° C. or at failure, when the cryogenic stress reached the tensile strength of the asphalt composition sample. A close-loop control system kept the specimen at constant length. The specimen were subjected to an increasing (cryogenic) tensile stress, due to the restrained thermal shrinkage by the close-loop control system. TSRST generally results in a temperature dependent function of cryogenic stress δ_{cry} (T) [MPa] in failure stress δ_{F} [MPa] and in failure temperature T_{f} [° C].

From the asphalt composition samples, the bituminous binder was extracted and recovered according to EN 12697-3.

The following binder properties are based on the European specifications EN 12591: a) penetration value at 25° C. in accordance with EN 1426, which reflects the consistency of the asphalt binder at ambient temperature where the higher value indicates a softer binder such that the higher the value the softer the binder; b) softening point (SP) temperature in accordance with EN 1427, which reflects the consistency of the asphalt binder at high temperature where the higher value indicates the amount of heat the asphalt binder needs in order to soften or to flow such that a higher value indicates the higher heat needed for the binder to soften or flow.

Further analysis may be conducted to measure rheological properties. The tests may be conducted using a Dynamic Shear Rheometer (DSR). For this test, a 10 mm plate is run at one frequency (10 rad/s) in a range of −30° C. to +90° C. These measurements are used to assess the performance of the binder at high, intermediate and low temperatures, thus providing an indication of asphalt composition performance pertaining to rutting resistance, stiffness and cracking susceptibility, respectively.

In addition to the DSR, bending beam rheometer (BBR) test may be used to evaluate the low temperature behavior of bituminous binders. The tests are typically conducted on beams of bituminous binder. A constant load is applied to the mid-point of the specimen with a defined load and time. The deflection of the beam is measured as a function of time. From the data obtained, the stiffness modulus and the log slope of the creep curve at test temperature are measured. The bituminous binders may be aged by rolling thin film oven (RTFO) and pressure aging vessel (PAV) before measuring the BBR. In this test, the binders are extracted from asphalt mixtures, hence it is chosen only to age with the PAV for 20 hours at 100° C. to prevent double short term aging.

### Example 2

As in Example 1, the asphalt mixture composition of the type Asphalt Concrete (AC) 16 (nominal maximum aggregate size) BN (base binder layer) (referred to herein as “AC16BN”) according to EN 13108-01 European standard for binder layers was employed for the asphalt composition samples, with formulation and gradation curve as shown in FIG. 2. The target binder content for the mix was 4.7%, a PEN 50/70 graded bitumen was used to adjust the binder properties.

In one of the examples, the reference asphalt mixture was modified by 70 wt. % of RA added to the total asphalt mixture composition. In another example, the test asphalt composition mixture included 70 wt. % of RA and included the bio-based additive SYLVAROAD™ RP1000 used in an amount of 4 wt. % per weight of binder content from the RA. Asphalt slabs were produced from the asphalt composition mixtures. Table 5 shows the composition of the asphalt mixtures. Of note, the compositions were similar to one another.

TABLE 5
Asphalt Compositions.

| | Binder | Air void | Bulk density |
|---|---|---|---|
| Asphalt Composition Mixtures | content (%) | (%) | P_{bssd} (Mg/m³) |
| Mixture 1 (0% RA, Tc = 135° C.) | 4.72 | 3.5 | 2.589 |
| Mixture 2 (70% RA, Tc = 135° C.) | 4.59 | 3.5 | 2.493 |
| Mixture 3a (70% RA + bio-based | 4.72 | 3.3 | 2.496 |
| additive, Tc = 135° C.) | | | |
| Mixture 3b (70% RA + bio-based | 4.87 | 3.4 | 2.495 |
| additive, Tc = 95° C.) | | | |

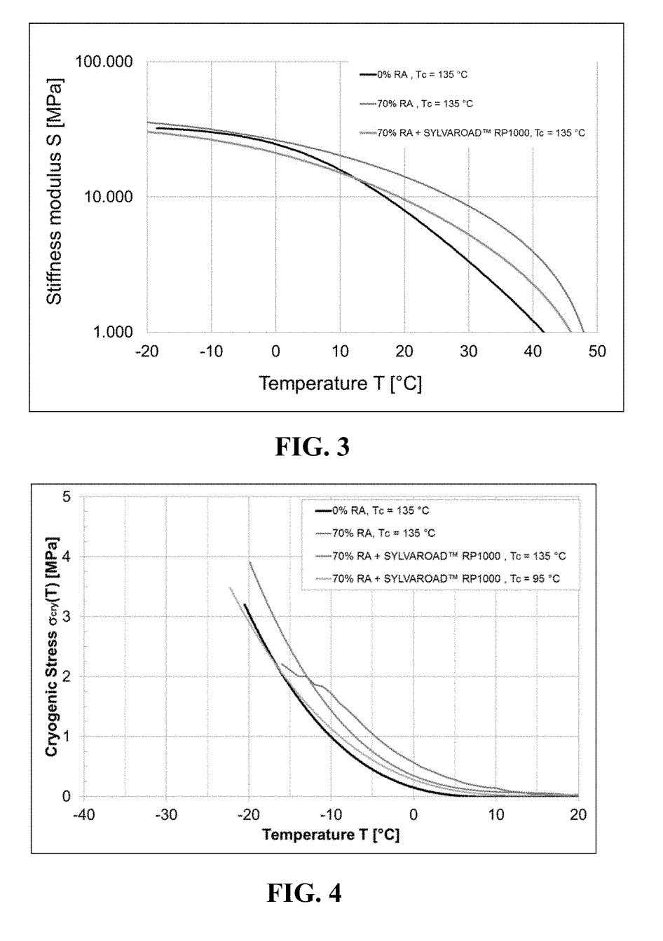

The stiffness modulus of the three asphalt composition mixtures compacted at 135° C. are shown in FIG. 3. The addition of 70 wt. % of RA to the asphalt composition increases the stiffness compared to the reference asphalt composition mixture not containing RA. This is represented in the higher stiffness modulus in the temperature range of 0 to 50° C. The stiffness modulus of the test asphalt compositions containing RA and rejuvenator may be 1000 MPa-30,000 MPa. When bio-based additive was added to the asphalt composition containing 70 wt. % RA, it was observed that especially the stiffness modulus between −20 to 10° C. was lower compared to the reference mixture. The stiffness modulus within the temperature range of 20 to 50° C. of the test asphalt containing 70 wt. % of RA and bio-based additive was higher compared to the reference asphalt composition mixture. This indicates a better resistance to low temperature cracking while retaining the benefits of RA for the rutting performance at higher temperatures compared to the reference mixture shows that the mixtures are somewhat similar.

The results of the Thermal Stress Restrain Specimen tests on the different asphalt mixtures are shown in Table 6.

TABLE 6
TSRST Results.

| | Failure temperature | Failure stress |
|---|---|---|
| Asphalt mixture | T_{F} (° C.) | δ_{F} (MPa) |
| Mixture 1 (0% RA, Tc = 135° C.) | −20.5 | 3.385 |
| Mixture 2 (70% RA, Tc = 135° C.) | −17.0 | 2.270 |
| Mixture 3a (70% RA + bio-based | −20.0 | 3.691 |
| additive, Tc = 135° C.) | | |
| Mixture 3b (70% RA + bio-based | −22.4 | 3.375 |
| additive, Tc = 95° C.) | | |

The bulk densities, as shown in Table 6, demonstrate that all 4 mixtures were equally compacted. Even for Mixture 3b where the compaction temperature was 40° C. lower compared to the other 3 mixtures. This result indicated that it is possible to obtain good compaction with 70 wt. % of RA present in asphalt composition mixtures in combination with rejuvenating agent(s) at lower temperatures.

The failure stress for Mixture 2 was lower compared to the other Mixtures, as Mixture 2 was constrained in deformation. This means the stiffness of the binder was higher, which is in line with stiffness modulus results. The bio-based additive lowers the stiffness and restores the modulus comparable to Mixture 1.

Compared to the asphalt composition mixture reference (0 wt. % RA), the addition of 70 wt. % of RA to the asphalt composition mixture resulted in weak low temperature performance. The failure temperature results are illustrated in FIG. 5 and indicate that the combination of 70 wt. % of RA with bio-based additive, or rejuvenator, restores the crack resistance comparable to the reference mixture. A similar explanation applies to the results of the Failure Stress results. The bio-based additive is able to restore the properties within the bitumen matrix in a way that the behavior of the virgin bitumen returns.

### Example 3

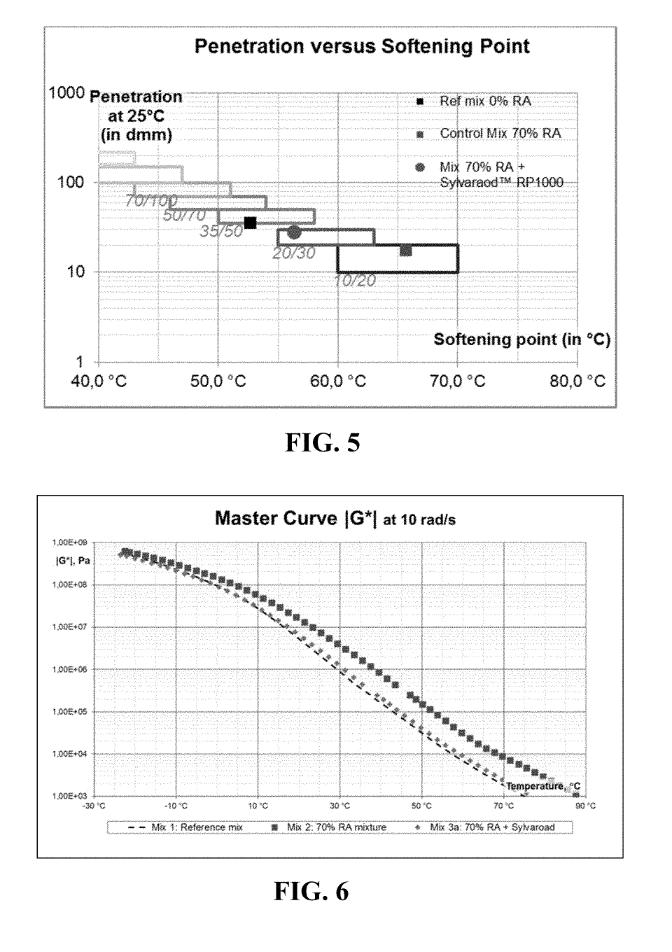

From all three asphalt composition mixtures, the binders were extracted and recovered according to EN 12697-3. The recovered binder was evaluated for conventional properties such as penetration and softening point. FIG. 6 illustrates the conventional properties. It can be seen that the binder extracted from the asphalt compositions having 70 wt. % RA are ‘hard’, while the binder extracted from the asphalt compositions having 70 wt. % of RA mixture together with additive/rejuvenator had improved properties similar to the reference mixture containing virgin materials without RA and/or rejuvenator.

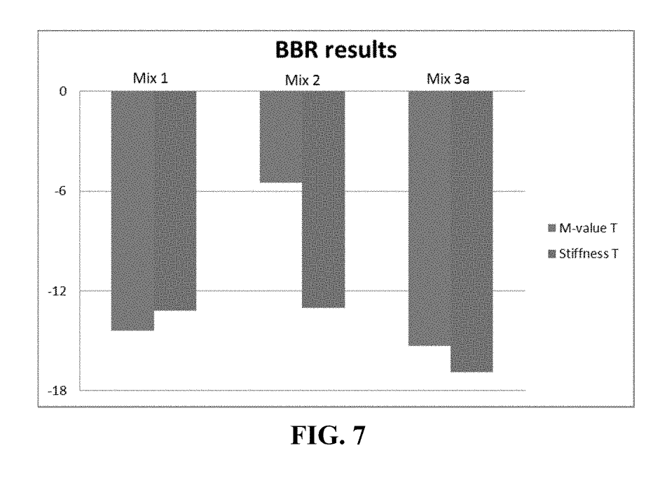

The effectiveness of the additive/rejuvenator over a broad temperature range may be best addressed using rheological behavior during shear tests (moduli and phase angle measurements). The measurements were made at a fixed frequency of 10 rad/s and in a temperature sweep from −30° C. to +90° C. This allowed the direct generation of master curves in a wide range of conditions without any shift. During the measurement, the data was controlled in order to remain in the linear elastic domain. The data was analyzed with |G*| vs. temperature at a fixed frequency as illustrated in FIG. 7.

FIG. 7 shows the rheological behavior of the four binders recovered from the reclaimed asphalt mixtures. Mixture 2 showed increased stiffness at higher temperatures compared to the reference mixture. This may be explained by the addition of 70 wt. % of RA. For Mixtures 3a, whereby the RA was treated with bio-based additive, the rheological behavior at 20° C. was similar to the reference mixture. At higher temperatures, the rheological properties due to the presence of RA were maintained, such as the stiffness properties.

Additionally, the low temperature behavior of the binders was tested with the bending beam rheometer. The extracted binders were first aged with the PAV at 100° C. for 20 hours. For BBR evaluation the following Mixtures were used: Mix 1; reference mixture, Mix 2; 70 wt. % RA mixture and Mix 3a; 70 wt. % RA mixture with bio-based additive.

The creep stiffness temperature was determined when the stiffness modulus was no more than 300 MPa. The m-value temperature was determined when the m-value was above 0.300. The m-value was determined by the slope of the logarithmic of the stiffness curves vs. the logarithmic of time.

Based on the results shown in FIG. 8, it can be seen that increased RA content in the asphalt mixture decreases the m-value, and slight stiffness remained. When the bio-based additive was added to the mix, both the m -value was restored and stiffness improved over both the reference mix as well as the 70 wt. % RA mixture. The bio-based additive recovered the relaxation of RA.

1. A method for evaluating an asphalt mix composition comprising:
providing a reference asphalt mix composition comprising aggregates and virgin bitumen in a ratio of 90:10 to 95:5 of aggregates to virgin bitumen;
providing a test asphalt mix composition comprising aggregates, a reclaimed asphalt and a rejuvenating agent;
optionally performing at least one of compaction test, creep stiffness modulus test and low temperature cracking susceptibility test;
performing a TSRST test on the test asphalt mix composition and the reference asphalt mix compositions to obtain failure temperature and failure stress data for all asphalt mix compositions;
comparing the failure temperature and failure stress data of the reference and test asphalt mix compositions to evaluate whether the test asphalt mix composition has an optimal amount of reclaimed asphalt, wherein the test asphalt mix composition with the optimal amount of reclaimed asphalt is defined as having a failure temperature within±5% of the failure temperature of the first reference asphalt mix composition, and a failure stress that is equal to or greater than the failure stress of the first reference asphalt mix composition.

2. The method of claim 1, wherein providing a reference asphalt mix composition comprises the aggregates and virgin bitumen in a ratio of 90:10 to 98:2 of aggregates to virgin bitumen.

3. The method of claim 1, further comprising performing a compaction test at a temperature of 90° C.-135° C.

4. The method of claim 1, further comprising performing a compaction test at a temperature of 90° C.-115° C.

5. The method of claim 1, wherein providing the reference asphalt mix composition comprises: providing an asphalt mix composition comprising aggregates, virgin bitumen, and a binder wherein the ratio of aggregates to virgin bitumen is in a ratio of 90:10 to 95:5.

6. The method of claim 5, further comprising recovering the binder from the reclaimed asphalt to produce a recovered binder.

7. The method of claim 6, further comprising performing a creep stiffness modulus test at a temperature less than 50° C. on the recovered binder to determine if the test asphalt mix composition has a stiffness modulus of less than 300 MPa and an m-value 0.300, wherein the m-value is determined by slope of the logarithmic of the stiffness curves versus logarithmic of time.

8. The method of claim 1, wherein providing the test asphalt composition comprises providing an asphalt mix composition comprising aggregates, a reclaimed asphalt and a rejuvenating agent, wherein the reclaimed asphalt is in an amount of at least 15 wt. % based upon the asphalt mix composition, and wherein the rejuvenating agent is in an amount of 0.1 wt. % to 20 wt. % based upon the combined amount of binder from reclaimed asphalt and rejuvenating agent.

9. The method of claim 1, further comprising:
optionally providing a control reference asphalt mix composition comprising aggregates and a reclaimed asphalt wherein the control reference asphalt composition does not contain added rejuvenating agent;
performing a TSRST test on the control reference asphalt mix composition to obtain failure temperature and failure stress data for the control reference asphalt mix composition;
comparing the failure temperature and failure stress data of the control reference asphalt mix composition and test asphalt mix composition to evaluate whether the test asphalt mix composition has a lower failure temperature and a higher failure stress compared to the failure temperature and the failure stress of the control reference asphalt mix composition.

10. The method of claim 9 wherein optionally providing the control reference asphalt mix composition comprises providing an asphalt mix composition comprising aggregates and a reclaimed asphalt wherein the reclaimed asphalt comprises a binder.

11. The method of claim 1, further comprising:
optionally providing a control reference asphalt mix composition comprising aggregates and a reclaimed asphalt wherein the control reference asphalt composition contains an added rejuvenating agent that is different from the rejuvenating agent in the test asphalt mix composition;
performing a TSRST test on the control reference asphalt mix composition to obtain failure temperature and failure stress data for the control reference asphalt mix composition;
comparing the failure temperature and failure stress data of the control reference asphalt mix composition and test asphalt mix composition to evaluate whether the test asphalt mix composition has a lower failure temperature and a higher failure stress compared to the failure temperature and the failure stress of the control reference asphalt mix composition.
